(19) United States
(12) Patent Application Publication
(10) Pub. No.: US 2011/0046205 A1
(43) Pub. Date: Feb. 24, 2011

(54) METHOD FOR DISSOLVING CHARGED NUCLEIC ACID IN AN ORGANIC LIQUID

(76) Inventors: Hans Kosak, Bonn (DE); Andre Josten, Nurnberg (DE)

Correspondence Address: FISH & RICHARDSON P.C. (TC), PO BOX 1022, MINNEAPOLIS, MN, 55440-1022, US

(21) Appl. No.: 12/377,769

(22) PCT Filed: Aug. 16, 2007
(86) PCT No.: PCT/EP07/07257

§ 371 Date: Dec. 22, 2009

(30) Foreign Application Priority Data
Jun. 8, 2007 (DE) 10 2006 038 791.0

(51) Int. Cl. A61K 31/7084; C07H 21/00; A61P 43/00; C10L 1/238; C10L 5/00; G01N 33/00; C12Q 1/68; G01N 33/02

(52) U.S. Cl. 514/44 R; 536/23.1; 44/313; 44/308; 44/605; 436/94; 435/6; 436/20

(57) ABSTRACT

The invention relates to a method for dissolving charged nucleic acids in an organic first liquid which is immiscible with water. The method comprises the following steps: a) providing a solution of the nucleic acids in an aqueous second liquid, b) precipitating the nucleic acids by adding a complexing agent to the second liquid, the complexing agent forming complexes with the nucleic acids that are insoluble in the second liquid, c) removing the complexes from the second liquid, d) dissolving the complexes in a third liquid which consists of an amphiphilic compound or which contains an amphiphilic compound, and e) mixing the third liquid with the first liquid.

The invention relates to a method for dissolving charged nucleic acid in a water-immiscible organic liquid. The invention additionally relates to a water-immiscible organic liquid which comprises a nucleic acid dissolved therein and complexed by a complexing agent. The invention further relates to a use of such a liquid for carrying out an identification method.

U.S. Pat. No. 5,665,538 discloses the addition of DNA to petroleum material. In this case, the DNA is formulated such that it can be dissolved in the petroleum material and essentially cannot be removed therefrom by washing with water. The method disclosed in U.S. Pat. No. 5,665,538 is used to monitor the transport of a material, especially of a petroleum product. The DNA can be removed from the petroleum product and detected by an amplification reaction. In order to dissolve the DNA in the petroleum product, the DNA can be combined with a hydrophobic hapten. Another possibility is to use a DNA which is chemically modified in such a way that it is hydrophobic. For this purpose, the DNA may include sulfonucleotides which comprise thiophosphates which are modified by suitable agents, such as iodoethanol. Alternatively, it is possible to use a methylated DNA.

Said methods have the disadvantage that the preparation of the DNA required therefor is very burdensome. It is additionally burdensome to transfer the DNA subsequently into the aqueous phase in order to be able to detect it.

In the case of a methylated DNA, this can take place for example by arranging a biotin molecule at one end thereof, so that the DNA can be bound by streptavidin and thereby isolated. In the case of a DNA coupled with a hydrophobic hapten, the DNA can be isolated by means of an antibody which is specific for the hapten.

EP 1 394 544 A1 discloses a method for mixing ribonucleic acid into water-insoluble media. In this case, a water-insoluble medium is initially dissolved in a solvent and then mixed with the nucleic acid which is dissolved in water. For example, the water-insoluble medium may be polystyrene and the solvent may be chloroform. Before the polystyrene dissolved in chloroform is mixed with the nucleic acid dissolved in water, an intermediate solution of 95% ethanol and acetone is added to the nucleic acid dissolved in water. The polystyrene solution obtained by the mixing comprises nucleic acid and can be used as anti-counterfeiting label for products. The disadvantages of this method are that it is relatively burdensome and polymers are necessary therefor. In addition, EP 1 394 544 A1 does not disclose how the nucleic acid present in the polymer can be removed again so that it can be detected.

It is an object of the present invention to indicate a favorable and non-burdensome method with which a charged nucleic acid can be dissolved in a water-immiscible organic liquid. It is further intended to indicate a water-immiscible organic liquid in which charged nucleic acid is dissolved, and a use of this liquid. The nucleic acid is to be dissolved in such a way that it can be recovered without difficulty from the organic liquid. The use of such liquids, for example in pharmaceutical compositions, and the provision of methods for analyzing food items are likewise among the objects of the invention.

A charged nucleic acid means a nucleic acid whose nucleotides are linked together by phosphodiester linkages, with the phosphate residues involved in the phosphodiester linkages being negatively charged, as is the case in naturally occurring DNA or RNA. Normally, nucleic acids are readily soluble in water owing to their charge, but not in water-immiscible organic liquids such as hydrocarbons. A water-immiscible organic liquid means an organic liquid for which more than 1 liter, preferably more than 10 liters, of water are required to dissolve 1 ml. The liquid is in this connection generally one of biological origin. It may for example be an oil or a fat or wax in the molten state which is of vegetable, mineral or animal origin. The liquid may also be a molten substance which is normally in the form of a solid at room temperature. Because of possible destruction of the nucleic acid by pyrolysis, the temperature of the liquid should not exceed 120° C., preferably 100° C., in particular 80° C. A nucleic acid is considered to be dissolved in the context of the invention when it cannot be centrifuged down by centrifugation at 15 000×g for 5 minutes.

The problem is solved by the features of claims 1, 2, 23, 41, 44 and 46-48. Expedient embodiments result from the features of claims 3 to 22, 24 to 40, and 42, 43 and 45.

The invention relates to a method for dissolving charged nucleic acids in a water-immiscible liquid, comprising mixing nucleic acids which are present with a complexing agent in an amphiphilic liquid with the water-immiscible liquid.

A further embodiment provides a method for dissolving charged nucleic acids in a water-immiscible organic first liquid comprising the following steps:

a) provision of a solution of the nucleic acids in an aqueous second liquid,
b) precipitation of the nucleic acids by adding to the second liquid a complexing agent which forms insoluble complexes with the nucleic acids in the second liquid,
c) removal of the complexes from the second liquid,
d) dissolution of the complexes in a third liquid which consists of an amphiphilic compound or comprises an amphiphilic compound, and
e) mixing of the third liquid with the first liquid.

The methods of the invention make it possible in a very simple and cost-effective manner to prepare a solution of charged nucleic acids in a water-immiscible organic first liquid. The nucleic acids can moreover be dissolved in very high concentration in the first liquid. It is essential for this purpose that the complexes are brought into contact with the amphiphilic compound. Without the amphiphilic compound it is possible to dissolve only relatively small amounts of the complexed nucleic acids in the first liquid.

The method of the invention makes it possible to label the first liquid with DNA for anti-counterfeiting identification. For example, petroleum or a petroleum product can be labeled thereby with a specific DNA, and its transport route can be monitored by identifying the DNA from a sample of the petroleum or petroleum product. The possibility of dissolving DNA in a high concentration in the first or third liquid allows a highly concentrated stock solution to be prepared, which solution is suitable for labeling a large amount of petroleum or petroleum product, for example a tanker load. The method of the invention further makes it possible to provide nucleic acids, in particular in high concentrations, for chemical reactions or for storage in water-immiscible organic liquids. It is possible for example to make chemical reactions of DNA in oil possible thereby. In addition, nucleic acids can be stored in the water-immiscible organic liquid without cooling and therefore transported over long distances without great complexity and without undergoing degradation, especially enzymatic. The reason for this is presumably that nucleic-acid degrading enzymes require an aqueous environment for their activity.

It is further possible thereby to formulate nucleic acids for pharmaceutical applications, such as, for example, siRNAs, for example as ointment. Such a formulation has the advantage of a very long shelflife.

The present invention therefore also relates to pharmaceutical compositions which comprise nucleic acids which are complexed with a complexing agent according to the invention and are present in an amphiphilic compound.

In a preferred embodiment, the invention relates to a pharmaceutical composition which comprises a water-immiscible organic liquid according to the present invention.

In a further preferred embodiment, the nucleic acid is RNA and particularly preferably siRNA.

The pharmaceutical compositions are used to administer nucleic acids in a stable form. The compositions of the invention can for example be in the form of granules, powders, tablets, capsules, syrup, suppositories, injections, emulsions, suspensions or solutions. The compositions of the invention can be formulated for various types of administration, for example for topical, oral, buccal, sublingual, transmucosal, rectal, subcutaneous, intrathecal, intravenous, intramuscular, intraperitoneal, nasal, intraocular or intraventricular administration.

In a preferred embodiment, the pharmaceutical compositions are designed for topical application. In a particularly preferred embodiment, the pharmaceutical composition comprising the complexed nucleic acid of the invention is in the form of an ointment.

For recovering the nucleic acid from the first liquid, the nucleic acid can be extracted with a water-miscible solvent in which a high concentration of salts is present, e.g. by shaking. It is possible to use for this purpose for example sodium acetate-saturated ethanol or SDS-saturated ethanol or butanol. In this case, the nucleic acid precipitates in the water-miscible solvent and, after removal of the water-miscible solvent, e.g. by centrifugation, can be taken up in water and for example detected by means of a PCR.

A further aspect of the invention thus relates to methods for isolating nucleic acids from a water-immiscible liquid.

In a first embodiment, the method for isolating nucleic acids from a water-immiscible liquid comprises steps in which
a) the nucleic acids are extracted from the liquid with a water-miscible solvent by bringing the water-immiscible liquid into contact with the water-miscible solvent, wherein the water-miscible solvent comprises one or more salt(s);
b) the water-miscible solvent is separated from the water-immiscible liquid; and
c) the nucleic acids are isolated from the water-miscible solvent.

In a second embodiment, the method for isolating nucleic acids from a water-immiscible liquid comprises the mixing of this liquid with a solvent which comprises one or more salt(s) and dissolves in the water-immiscible liquid, whereby the nucleic acids precipitate.

A further embodiment relates to a method for isolating nucleic acids from a water-immiscible liquid, in which one or more salt(s) is (are) added to this liquid, whereby the nucleic acids precipitate.

Examples of suitable salts are sodium bromide, sodium dodecyl sulfate (SDS) or sodium acetate.

The salts are employed in an amount which corresponds to 50%, preferably 60%, more preferably 70%, particularly preferably 80% and most preferably 90% of the saturation solubility, i.e. which corresponds to the amount of the salt which must be employed under standard conditions in order to result in a saturated solution with the selected solvent. This amount can easily be ascertained by a person skilled in the art.

In a further preferred embodiment, the solutions are employed in the form of saturated solutions.

Water-miscible solvents which can be employed in the abovementioned first embodiment are thus for example saturated salt solutions in alcohols which are completely miscible with water, e.g. a saturated NaBr solution in ethanol.

Isolation of a nucleic acid from a water-immiscible liquid can be employed in a further embodiment of the invention in order to analyze food items. It is thus possible to isolate even tiny amounts of nucleic acids from food items and thereby to determine the origin of the constituents of the food item. Thus, for example, various types of oil can be identified on the basis of the nucleic acids found in the oils.

It has surprisingly been found that nucleic acids can also be isolated from samples of food items assumed not to contain any nucleic acids. Thus, in the context of the present invention, for example nucleic acids have been found in conventional edible oils such as rapeseed oil, olive oil, sunflower oil, pumpkin seed oil, sesame oil, grapeseed oil, walnut oil, safflower oil and palm oil.

It is possible by amplifying these nucleic acids to determine which constituents were present in the food item. Thus, for example, high-value edible oils such as, for example, olive oils can be distinguished from oils of less value.

The invention thus also relates to methods for analyzing food items in which
a) nucleic acids are isolated from one or more water-immiscible constituent(s) of the food item by using one of the abovementioned methods; and
b) the nucleic acids are analyzed.

In a preferred embodiment, the nucleic acids are analyzed by PCR.

The complexing agent is preferably a cationic detergent or an organic amine, in particular a quaternary amine. The quaternary amine is preferably cetyltrimethylammonium bromide (CTAB). CTAB is very suitable for the intended complexation and very reasonably priced.

The complexing agent is preferably added to the second liquid in step b) in dissolved form. This makes possible faster precipitation than on addition of an undissolved complexing agent.

The nucleic acids are preferably synthetically prepared nucleic acids having a known sequence. This allows a virtually unlimited number of codings to be provided for anti-counterfeiting labeling. It is particularly preferred for this purpose when the nucleic acids used for identification are “hidden” in a large number of further nucleic acids dissolved in the first liquid, so that the nucleic acids which are specifically serving for labeling cannot be detected by a sequencing. The further nucleic acids can be provided for example by using herring sperm DNA.

The nucleic acids can each have a chain length of from 5 to 100 nucleotides, preferably 10 to 80 nucleotides, particularly preferably 15 to 60 nucleotides. The nucleic acids may be DNA, in particular antisense DNA, or RNA, in particular siRNA. It is particularly advantageous for a therapeutic application if the nucleic acids comprise at least a part of a sequence of a human gene.

The nucleic acids may have a single-stranded or double-stranded configuration. A nucleic acid with a double-stranded configuration can be present in the form of two separate single strands or as hairpin loop structure.

The removal of the complexes in step c) preferably takes place by centrifugation or filtration. These methods represent a particularly simple and efficient way of removing the precipitated complexes.

The amphiphilic compound is preferably an organic solvent. The amphiphilic compound preferably comprises at least one ether group and/or at least one hydroxyl group.

Amphiphilic compounds which are particularly suitable as solvents can be described by the formula HO—R1-O—R2. R1 and R2 therein is in each case a hydrocarbon residue having 1 to 100 carbon atoms. The amphiphilic compound may be ethylene glycol monobutyl ether, ethylene glycol monoethyl ether or ethylene glycol monomethyl ether.

The complexes are preferably dissolved in the third liquid in an amount such that a nucleic acid concentration in the third liquid which is greater than 0.1 mg/ml, in particular greater than 1 mg/ml, preferably greater than 10 mg/ml, results therefrom. The first liquid may be a vegetable or animal oil or fat. The first liquid may be a liquid which is in the form of a solid at 20° C. It may be for example a wax.

The first liquid may also be a fuel for an internal combustion engine. The first liquid may be a mineral fat, a mineral oil or a mineral oil distillate or mineral oil distillate residue, in particular diesel fuel, light oil, heavy oil, toluene, benzene or gasoline.

The invention further relates to a water-immiscible organic liquid which comprises, dissolved therein, charged nucleic acids complexed by a complexing agent, and an amphiphilic compound. The nucleic acids are in this case synthetically prepared nucleic acids having a known sequence. Such a liquid can be prepared by the method of the invention. The liquid is particularly suitable for carrying out a method for detecting the nucleic acids for identifying the liquid. Identification of the liquid is particularly reliable if the previously known sequence of the nucleic acid is confirmed.

Advantageous embodiments of the invention are evident from the following exemplary embodiments.

Unless indicated otherwise, all the chemicals were obtained from Sigma-Aldrich Chemie GmbH, Eschenstrasse 5, 82024 Taufkirchen, Germany.

1. Precipitation of DNA using CTAB as complexing agent:

10 g of herring sperm DNA (HS-DNA) were dissolved in one liter of TE (10 mmol/l Tris HCl, 1 mmol/l EDTA, pH 8 in deionized water). 100 ml of a CTAB solution (100 mmol/l CTAB in deionized water) was added while stirring. The precipitate formed (CTAB-HS-DNA) was centrifuged down and dried at room temperature.

In addition, 1 μmol (equivalent to about 20 mg) of a synthetically prepared single-stranded DNA which was 62 nucleotides long and corresponded to sequence No. 1 in the appended sequence listing (labeling DNA, L-DNA) was dissolved in 1 ml of TE. 100 μl of the CTAB solution were added while stirring. The precipitate formed (CTAB-L-DNA) was centrifuged down and dried at room temperature.

To prepare a precipitate comprising both HS-DNA and L-DNA, 1 g of HS-DNA and 1 μmol of L-DNA were dissolved in 100 ml of TE. 10 ml of the CTAB solution were added while stirring. The precipitate formed (CTAB-HS-L-DNA) was centrifuged down and dried at room temperature.

2. Solubility of the DNA complexed using CTAB in water-immiscible liquids:

Approx. 1 mg portions of CTAB-HS-DNA were transferred into 1.4 ml Eppendorf reaction vessels. 1 ml portions of gasoline (BP super gasoline), diesel fuel (BP), mineral oil, sunflower oil (supermarket), rapeseed oil (supermarket), wheat germ oil (supermarket) or olive oil (supermarket) were added to each of the Eppendorf reaction vessels. The reaction vessels were then shaken vigorously and subsequently centrifuged at 15 000×g for 5 minutes. A precipitate was to be observed in all the reaction vessels.

3. Solubility of the DNA complexed using CTAB in water-immiscible liquids after the complexed DNA has been predissolved in alcohol:

1 g of CTAB-HS-DNA was dissolved in 50 ml of 1-butanol. 100 μl portions of the solution were transferred into 1.4 ml Eppendorf reaction vessels. 1 ml portions of gasoline, diesel, mineral oil, sunflower oil, rapeseed oil, wheat germ oil or olive oil were added to each of the Eppendorf reaction vessels. The reaction vessels were then shaken vigorously and subsequently centrifuged at 15 000×g for 5 minutes. A precipitate was to be observed in all the reaction vessels.

4. Solubility of the DNA complexed using CTAB in an amphiphilic compound:

Approx. 20 mg portions of CTAB-HS-DNA were transferred into 1.4 ml Eppendorf reaction vessels. 1000 μl, 500 μl, 200 μl, 100 μl, 50 μl or 20 μl portions of ethylene glycol monobutyl ether (EGE) were added to each of the Eppendorf reaction vessels. The reaction vessels were then shaken vigorously and subsequently centrifuged at 15 000×g for 5 minutes. No precipitate was to be observed in any of the reaction vessels. The CTAB-HS-DNA was completely dissolved.

5. Solubility of the DNA complexed using CTAB in a water-immiscible liquid after the complexed DNA has been predissolved in an amphiphilic compound:

2 g of CTAB-HS-DNA were dissolved in 50 ml of EGE. 100 μl and 500 μl portions of the solution were transferred into 2 ml Eppendorf reaction vessels. Respectively 900 μl and 500 μl portions of gasoline, diesel, mineral oil, sunflower oil, rapeseed oil, wheat germ oil or olive oil were added to each of the Eppendorf reaction vessels. The reaction vessels were then vigorously shaken and subsequently centrifuged at 15 000×g for 5 minutes. No precipitate was to be observed in any of the reaction vessels. The CTAB-HS-DNA was completely dissolved.

6. Extraction of DNA dissolved in water-immiscible liquids: 20 g of CTAB-HS-L-DNA were dissolved in 1 ml of EGE. 100 μl portions thereof were dissolved in 100 ml of gasoline, diesel, mineral oil, sunflower oil or rapeseed oil. To tract the DNA, 1 ml portions of the resulting solution were transferred into a 1.4 ml Eppendorf reaction vessel. 50 μl portions of SDS-saturated ethanol were added thereto, shaken vigorously and centrifuged at 15 000×g for 5 minutes. The supernatant was discarded. 1 ml portions of heptane were added to the resulting pellet. The reaction vessel was then shaken vigorously and again centrifuged at 15 000×g for 5 minutes. The supernatant was discarded and the pellet was taken up in 1 ml of 0.1 TE (1 part of TE, 9 parts of deionized water).

A polymerase chain reaction (PCR) was carried out with 2.5 μl portions of the aqueous solution of HS-DNA and L-DNA obtained in this way. The synthetically prepared L-DNA-specific primers 1 and 2 which correspond to sequences Nos 2 and 3 in the appended sequence listing were used for this purpose. The PCR was carried out in a total volume of 25 μl using a kit (peqGOLD PCR-Master-Mix S) from Peqlab (Carl-Thiersch-Str. 2b, 91052 Erlangen, Germany). The primer concentration was in each case 200 nm per primer, and 30 cycles were carried out with an annealing temperature of 55° C. Analysis took place by gel electrophoresis on 10% (w/v) polyacrylamide gels and subsequent silver staining. The L-DNA was detectable in all the samples. Extracts from gasoline, diesel, mineral oil, sunflower oil and rapeseed oil to which no DNA had been added were used as control. No L-DNA was detectable in the controls.

7. Stability of DNA dissolved in water-immiscible liquids:

20 mg of CTAB-HS-L-DNA were dissolved in 1 ml of EGE. 100 μl portions thereof were dissolved in 100 ml of diesel fuel, mineral oil, sunflower oil or rapeseed oil. 1 ml samples of each were taken and incubated at 4° C., room temperature, 40° C. and 80° C. for 24 hours. To extract the DNA, 50 μl portions of SDS-saturated ethanol were added to the samples, shaken vigorously and centrifuged at 15 000×g for 5 minutes. The supernatant was discarded. 1 ml portions of heptane were added to the resulting pellet, shaken vigorously and centrifuged at 15 000×g for 5 minutes. The supernatant was discarded and the pellet was taken up in 1 ml of 0.1 TE. A polymerase chain reaction (PCR) was carried out with 2.5 μl portions of the aqueous solution of HS-DNA and L-DNA obtained in this way. The synthetically prepared primers 1 and 2 were used for this purpose. The PCR was carried out in a total volume of 25 μl with the abovementioned kit from Peqlab. The primer concentration was in each case 200 nm per primer and 30 cycles were carried out with an annealing temperature of 55° C. Analysis took place by gel electrophoresis on 10% (w/v) polyacrylamide gels and subsequent silver staining. The L-DNA was detectable in all the samples. Extracts from gasoline, diesel, mineral oil, sunflower oil and rapeseed oil to which no DNA had been added were used as control. No L-DNA was detectable in the controls. The experiment shows that DNA dissolved in water-immiscible liquid is stable over a long period even without cooling and even at elevated temperature.

8. Introduction of DNA complexed by CTAB and predissolved in EGE into solids:

10 g of coconut fat (supermarket) were liquefied by heating. After cooling to about 50° C., 100 μl of 2% (w/v) CTAB-HS-L-DNA in EGE were added and mixed. The fat was solidified by cooling to 4° C.

9. Extraction of DNA from diesel/gasoline/rapeseed/sunflower oil without added DNA

100 μl portions of SDS-saturated ethanol were added at room temperature to 2 samples each of 1 ml of diesel, gasoline, rapeseed oil and sunflower oil in 1.4 ml plastic reaction tubes, and the samples were shaken for 30 seconds and then incubated at 4° C. for 1 h. To separate the phases, the samples were centrifuged at 4° C. and max. acceleration (approx. 16 000×g) for 15 min. A slight pellet was visible in all the samples. The liquid phase was taken off and the pellet was mixed with 1 ml of n-heptane. The samples were mixed for about 20-30 sec and then centrifuged at 4° C. and max. acceleration (approx. 16 000×g) for 2 min. The supernatant was taken off and discarded. The reaction tubes were inverted and left to stand for about 10-20 min to dry the pellet. The pellet was dissolved in 100 μl of 0.1×TE with 0.02% Tween 20. The DNA content in the samples was determined by means of the PicoGreen assay using a lambda DNA calibration plot and carried out in accordance with the manufacturer's information (PicoGreen® dsDNA Quantitation Kit, Catalog Number-P11496, Invitrogen GmbH, Technologiepark Karlsruhe, Emmy-Noether Strasse 10, 76131 Karlsruhe). Nucleic acid was detectable in all the samples. The concentration was between 1 and 7 ng/ml (see table 1).

TABLE 1
DNA content in various types of oil

| | DNA content in |
|---|---|
| Sample | ng/ml |
| Gasoline 1 | 2.7 |
| Gasoline 2 | 2.4 |
| Diesel 1 | 6.9 |
| Diesel 2 | 6.8 |
| Rapeseed 1 | 2.4 |
| Rapeseed 2 | 3.0 |
| Sunflower 1 | 1.3 |
| Sunflower 2 | 1.6 |

1. A method for dissolving nucleic acids in a water-immiscible liquid comprising mixing nucleic acids which are present with a complexing agent in an amphiphilic liquid with the water-immiscible liquid.

2. A method for dissolving charged nucleic acids in a water-immiscible organic first liquid comprising the following steps:
a) provision of a solution of the nucleic acids in an aqueous second liquid,
b) precipitation of the nucleic acids by adding to the second liquid a complexing agent which forms insoluble complexes with the nucleic acids in the second liquid,
c) removal of the complexes from the second liquid,
d) dissolution of the complexes in a third liquid which consists of an amphiphilic compound or comprises an amphiphilic compound, and
e) mixing of the third liquid with the first liquid.

3. The method as claimed in claim 1 or 2, wherein the complexing agent is a cationic detergent or an organic amine, in particular a quaternary amine.

4. The method as claimed in claim 3, wherein the quaternary amine is cetyltrimethylammonium bromide (CTAB).

5. The method as claimed in any of claims 2-4, wherein the complexing agent is added to the second liquid in step b) in dissolved form.

6. The method as claimed in any of the preceding claims, wherein the nucleic acids are synthetically prepared nucleic acids having a known sequence.

7. The method as claimed in any of the preceding claims, wherein the nucleic acids each have a chain length of from 5 to 100 nucleotides, preferably 10 to 80 nucleotides, particularly preferably 15 to 60 nucleotides.

8. The method as claimed in any of the preceding claims, wherein the nucleic acids consist of DNA or of RNA.

9. The method according to that of the preceding claims, wherein the nucleic acids are antisense DNAs or siRNAs.

10. The method according to that of the preceding claims, wherein the nucleic acids comprise at least a part of a sequence of a human gene.

11. The method as claimed in any of the preceding claims, wherein the nucleic acids have a single-stranded or double-stranded configuration.

12. The method as claimed in any of the preceding claims, wherein the removal of the complexes in step c) takes place by centrifugation or filtration.

13. The method as claimed in any of the preceding claims, wherein the amphiphilic compound is an organic solvent.

14. The method as claimed in any of the preceding claims, wherein the amphiphilic compound comprises at least one ether group.

15. The method as claimed in any of the preceding claims, wherein the amphiphilic compound comprises at least one hydroxyl group.

16. The method as claimed in any of the preceding claims, wherein the amphiphilic compound can be described by the formula HO—R1-O—R2, wherein R1 and R2 is in each case a hydrocarbon residue having 1 to 100 carbon atoms.

17. The method as claimed in any of the preceding claims, wherein the amphiphilic compound is ethylene glycol monobutyl ether, ethylene glycol monoethyl ether or ethylene glycol monomethyl ether.

18. The method as claimed in any of the preceding claims, wherein the complexes are dissolved in the third liquid in an amount such that a nucleic acid concentration in the third liquid which is greater than 0.1 mg/ml, in particular greater than 1 mg/ml, preferably greater than 10 mg/ml, results therefrom.

19. The method as claimed in any of the preceding claims, wherein the first liquid is a vegetable or animal oil or fat.

20. The method as claimed in any of the preceding claims, wherein the first liquid is a solid, in particular a wax, at 20° C.

21. The method as claimed in any of claims 1 to 18, wherein the first liquid is a fuel for an internal combustion engine.

22. The method as claimed in any of the preceding claims, wherein the first liquid is a mineral fat, a mineral oil or a mineral oil distillate or distillate residue, in particular diesel fuel, light oil, heavy oil, toluene, benzene or gasoline.

23. A water-immiscible organic liquid comprising, dissolved therein, charged synthetically prepared nucleic acids complexed by a complexing agent and having a known sequence, and an amphiphilic compound.

24. The liquid as claimed in claim 23, wherein the complexing agent is a cationic detergent or an organic amine, in particular a quaternary amine.

25. The liquid as claimed in claim 24, wherein the quaternary amine is cetyltrimethylammonium bromide (CTAB).

26. The liquid as claimed in any of claims 23 to 25, wherein the nucleic acids each have a chain length of from 5 to 100 nucleotides, preferably 10 to 80 nucleotides, particularly preferably 15 to 60 nucleotides.

27. The liquid as claimed in any of claims 23 to 26, wherein the nucleic acids consist of DNA or of RNA.

28. The liquid as claimed in any of claims 23 to 27, wherein the nucleic acids are antisense DNAs or siRNAs.

29. The liquid as claimed in any of claims 23 to 28, wherein the nucleic acids comprise at least one part of a sequence of a human gene.

30. The liquid as claimed in any of claims 23 to 29, wherein the nucleic acids have a single-stranded or double-stranded configuration.

31. The liquid as claimed in any of claims 23 to 30, wherein the amphiphilic compound is an organic solvent.

32. The liquid as claimed in any of claims 23 to 31, wherein the amphiphilic compound comprises at least one ether group.

33. The liquid as claimed in any of claims 23 to 32, wherein the amphiphilic compound comprises at least one hydroxyl group.

34. The liquid as claimed in any of claims 23 to 33, where the amphiphilic compound can be described by the formula HO—R1-O—R2, wherein R1 and R2 is in each case a hydrocarbon residue having 1 to 100 carbon atoms.

35. The liquid as claimed in any of claims 23 to 34, wherein the amphiphilic compound is ethylene glycol monobutyl ether, ethylene glycol monoethyl ether or ethylene glycol monomethyl ether.

36. The liquid as claimed in any of claims 23 to 35, wherein the nucleic acid is present therein in a concentration which is greater than 0.1 mg/ml, in particular greater than 1 mg/ml, preferably greater than 10 mg/ml.

37. The liquid as claimed in any of claims 23 to 36, wherein the liquid is a vegetable or animal oil or fat.

38. The liquid as claimed in any of claims 23 to 37, wherein the liquid is a solid, in particular a wax, at 20° C.

39. The liquid as claimed in any of claims 23 to 37, wherein the liquid is a fuel for an internal combustion engine.

40. The liquid as claimed in any of claims 23 to 39, wherein the liquid is a mineral fat, a mineral oil or a mineral oil distillate or distillate residue, in particular diesel fuel, light oil, heavy oil, toluene, benzene or gasoline.

41. The use of a liquid as claimed in any of claims 23 to 40 for carrying out a method for detecting the nucleic acids to identify the liquid.

42. The use as claimed in claim 41, wherein the sequence of the nucleic acids is identified.

43. The use as claimed in claim 42, wherein the identification of the sequence of the nucleic acids takes place by sequencing, hybridization or carrying out a polymerase chain reaction (PCR).

44. A pharmaceutical composition which comprises a liquid as claimed in any of claims 23-40.

45. The pharmaceutical composition as claimed in claim 44, wherein the composition is designed for topical application.

46. A method for isolating nucleic acids from a water-immiscible liquid comprises steps in which:
a) the nucleic acids are extracted from the liquid with a water-miscible solvent by bringing the water-immiscible liquid into contact with the water-miscible solvent, wherein the water-miscible solvent comprises one or more salt(s);
b) the water-miscible solvent is separated from the water-immiscible liquid; and
c) the nucleic acids are isolated from the water-miscible solvent.

47. A method for isolating nucleic acids from a water-immiscible liquid which comprises the mixing of this liquid with a solvent which comprises one or more salt(s) and dissolves in the water-immiscible liquid, whereby the nucleic acids precipitate.

48. A method for analyzing food items, which comprises steps in which
a) nucleic acids are isolated from one or more water-immiscible constituent(s) of the food item by using one of the methods as claimed in claims 46 or 47; and
b) the nucleic acids are analyzed.


SEQUENCE LISTING (3 sequences)

SEQ ID NO: 1 - DNA, 62 bp, Artificial Sequence
  Oligonucleotide
gatcctgcataatggtagtagtcggccggtacgtatcacctatacgatgtcagcagattatg

SEQ ID NO: 2 - DNA, 22 bp, Artificial Sequence
  Oligonucleotide
cataatctgctgacatcgtata

SEQ ID NO: 3 - DNA, 21 bp, Artificial Sequence
  Oligonucleotide
gatcctgcataatggtagtag